(19) United States
(12) Patent Application Publication
(10) Pub. No.: US 2015/0320339 A1
(43) Pub. Date: Nov. 12, 2015

(54) SYSTEM AND METHOD FOR ANALYZING PATIENT ORIENTATION, LOCATION AND MOVEMENT

(71) Applicant: Centauri Medical Inc., Newark, CA (US)

(72) Inventors: Barrett J. LARSON, Palo Alto, CA (US); Daniel Z. Shen, Stanford, CA (US)

(21) Appl. No.: 14/244,720

(22) PCT Filed: Oct. 3, 2012
(86) PCT No.: PCT/US12/00488

§ 371 Date: Apr. 3, 2014

Related U.S. Application Data
(60) Provisional application No. 61/542,785, filed on Oct. 3, 2011.

(51) Int. Cl. A61B 5/11; A61B 5/00

(52) U.S. Cl. CPC A61B 5/1114; A61B 5/1115; A61B 5/1117; A61B 5/7275

(57) ABSTRACT

Systems, devices and methods for analyzing the movement, orientation, and location of patients and other users within a hospital, nursing home, or other setting where patient monitoring is desired. One or more sensors or markers are positioned on the body of the user, and a plurality of readers or transceivers in known relative location within the patient care environment detect or communicate with the sensors or markers to identify user location. Location sensing techniques include multi-angulation, multi-lateration, scene analysis (such as visual image analysis), or proximity. Analysis techniques used include time-of-flight analysis, signal strength analysis, image, and video analysis.

### RELATED APPLICATIONS

This application claims the benefit of the following U.S. patent applications: This application is the National Phase of PCT Application Number PCT/US2012/000488 which in turn is a conversion of Ser. No. 61/542,785, filed Oct. 3, 2011; and is a continuation-in-part of Ser. No. 13/070,189, filed Mar. 23, 2011, which is a conversion of U.S. Patent Application Ser. No. 61/326,664, filed Apr. 22, 2010, entitled Methods and Devices that Enable the Sensing of Body Surface Markers for the Prevention and Treatment of Pressure Ulcers and Other Wounds; Ser. No. 61/411,647, filed Nov. 9, 2010, entitled Method and Device for Surface Pressure Monitoring; Ser. No. 61/393,364, filed Oct. 15, 2010, entitled Patient Position, Orientation, and Surface Pressure Monitoring Device; and Ser. No. 61/373,260, filed Aug. 12, 2010, entitled Sensing System that Automatically Identifies and Tracks Body Surface Markers to Allow for the Delivery of Targeted Therapy. The foregoing are all assigned to the same assignee as the present invention, and are all incorporated herein by reference for all purposes.

### FIELD OF THE INVENTION

This invention relates generally to patient monitoring systems and methods, and more particularly relates to systems and methods for detecting and analyzing patient orientation, location and movement

### BACKGROUND OF THE INVENTION

Many situations exist in which it is desirable to detect and understand the location, orientation and movement of a person, for example a patient in a hospital or care home. Bed exits and falls represent significant health risks for individuals who, for one reason or another, have limited mobility and yet cannot be constantly monitored visually.

While complicated systems exist in the prior art that provide some indication of location, for example pressure mats, there has been a need for a simple, efficient, reliable and cost-effective method for detecting location, orientation, and/or movement of patients to, among other things, to alert a caregiver to, for example, a bed exit, a fall, or live tracking of a patient within a care environment.

### SUMMARY OF THE INVENTION

The present invention is directed to systems, methods and devices for analyzing the movement, orientation, and location of patients and other users within a hospital, nursing home, or other setting where patient monitoring is desired. While the invention can be used for monitoring patients and other users of all types, for purposes of simplicity and clarity herein, the term “patient” will be used to encompass all such users both in the Specification and in the claims.

Depending upon the embodiment, the present invention uses one or more of several methods of location sensing. On a broad level, the location sensing modality used can include multi-angulation, multi-lateration, scene analysis (such as visual image analysis), or proximity sensing. Within each category, different methods are used such as time-of-flight analysis, also referred to as time-delay, signal strength analysis, image, and video analysis. Different sensing signals can also be used, such as electromagnetic radiation, sound, magnetic sensing, and barometric pressure sensing, physical distance sensing. Currently several techniques allow for centimeter or millimeter/sub-millimeter level accuracy of location sensing, many of which are 3D location sensing. These include radiofrequency or magnetic sensing using received signal strength (RSSI), or image/video based 3D location sensing, though other technologies for location sensing will be apparent to those skilled in the art given the teachings herein. The apparatus and methods described herein may use one or more of these location sensing technologies, but can use any location sensing technology that provides sufficient resolution. In one embodiment of the present invention described herein, the apparatus of the invention uses a radiofrequency RSSI location sending system to determine the location, movement, and 3D orientation of a patient sensor.

The various objects of the invention can be better appreciated from the following Detailed Description, taken together with the appended Figures.

### THE FIGURES

FIG. 1 illustrates the various axes for defining movement of the human body.

FIGS. 2A-2C illustrate some possible options for the placement of sensors on the body for embodiments of the invention which utilize a plurality of sensors.

FIGS. 3A-3B illustrate some possible options for the placement of a single sensor on the body for embodiments of the invention which utilize a single sensor.

FIG. 4 illustrates an embodiment of the invention showing a single sensor on the patient, with a plurality of transceivers in known locations in communication with a host.

FIG. 5 illustrates the logical arrangement of signal processing for the embodiment of FIG. 4.

FIG. 6 illustrates in an alternative manner the logical arrangement of signal processing for the embodiment of FIG. 4.

FIG. 7 illustrates an embodiment of a sensor in accordance with aspects of the invention discussed in connection with FIG. 6, although the particular features can be provided by one or more sensors.

FIG. 8 illustrates an embodiment of a sensor having indicia for assisting in placement of the device on the patient in a predetermined orientation.

### DETAILED DESCRIPTION OF THE INVENTION

Location-Based Orientation Sensing

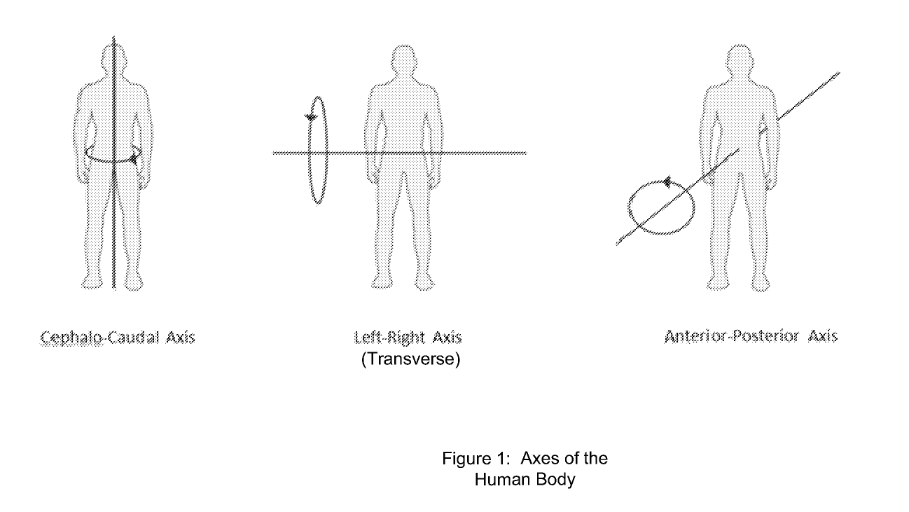

Referring first to FIG. 1, the various axes of motion for the human body are illustrated for reference purposes herein. The cephalo-caudal axis characterizes rotation around a line extending through the center of the body from the head to the feet. The transverse axis is parallel to the ground for a standing patient and divides the body into relatively cephalo and relatively caudal portions. The anterior-posterior axis extends from the back to the front of an individual and is parallel to the ground for a standing patient. Each of these axes can be used to measure one component of the patient's orientation.

The method and apparatus of the present invention can be used to measure the location, 3-dimensional orientation, and movements of a user. This information can be used to provide an indication of a patient's location, orientation, cardiorespiratory parameters (such as heart and breathing rate), neurologic status (such as seizures), falls, and bed exits, among other things.

In certain cases, such as for pressure ulcer management, it is important to know when the user is rotated on their right side, left side or back side and it may be useful to know their approximate or precise angle of rotation. Another component of the orientation that can be measured is the tilt of the user, i.e. rotation about the transverse axis. The user's torso may be tilted up relative to his/her feet, such as in reverse-Trendelenberg position, or the feet may be tilted up relative to the torso, such as in Trendelenberg position. The user's torso may also be tilted up at a different angle relative to the legs, such as when the head of a hospital bed is lifted.

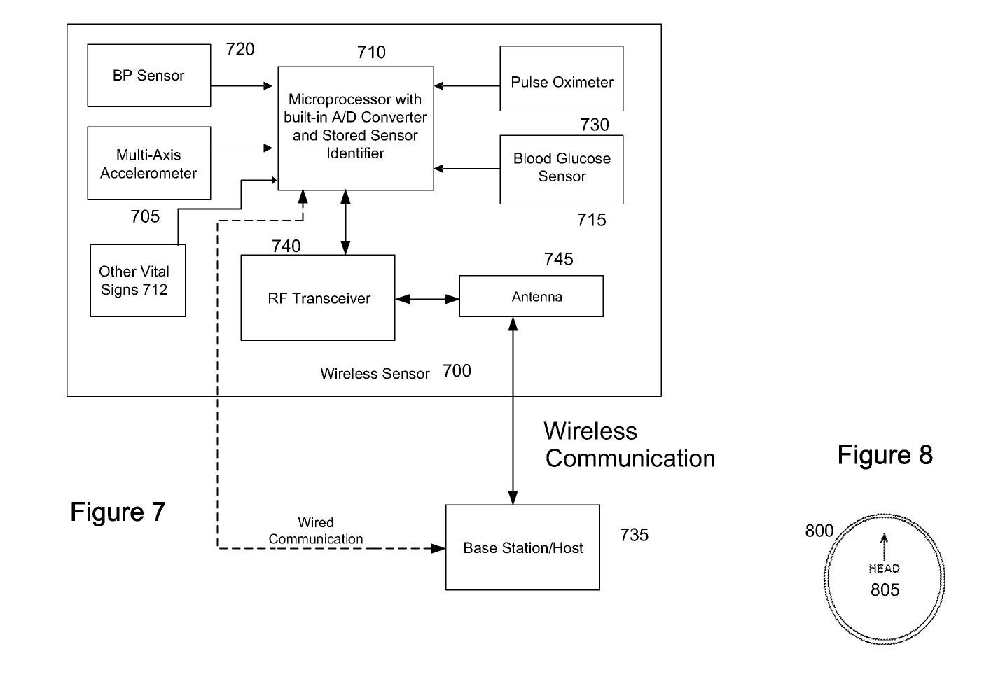

Sensors can be placed directly on, near, or at a distance from the user's body, as long as the relationship between the sensor and the user's body (or a portion of the user's body) is known or can be determined. If the relationship between the sensor and the user's body is known, then the location, orientation, and movements of the sensor will directly reflect the location, orientation, and movements of the monitored user. It should be noted that one or more sensors can be used to determine the orientation, location, and movements of the user. Suitable sensors and related information are described in U.S. patent application Ser. No. 13/070,189, filed 23 Mar. 2011, and more specifically as discussed hereinafter in connection with FIGS. 7 and 8.

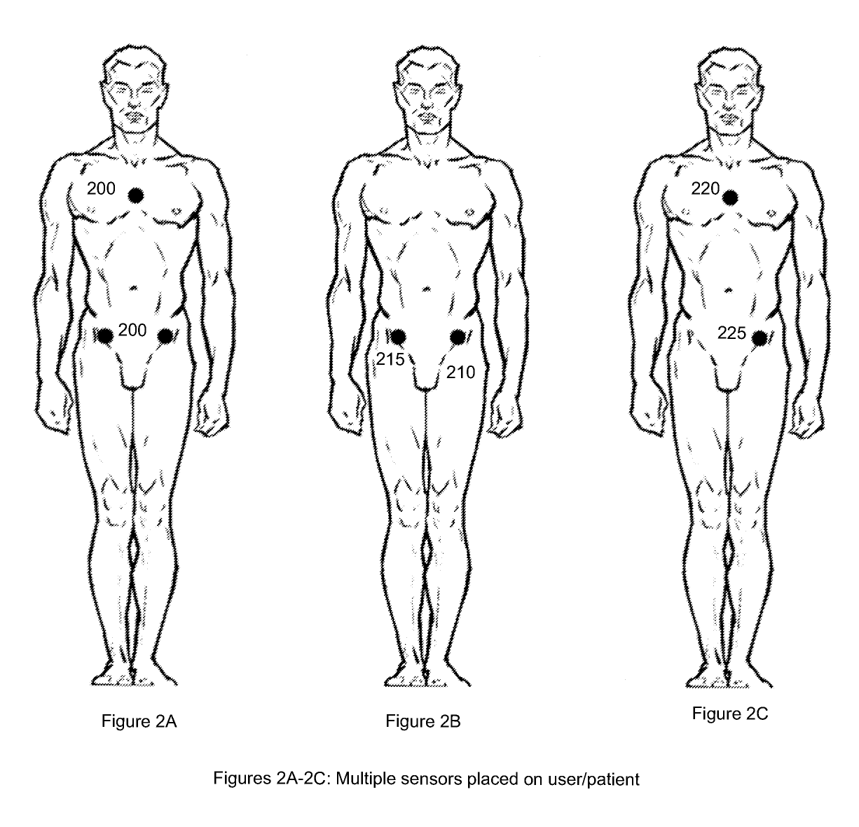

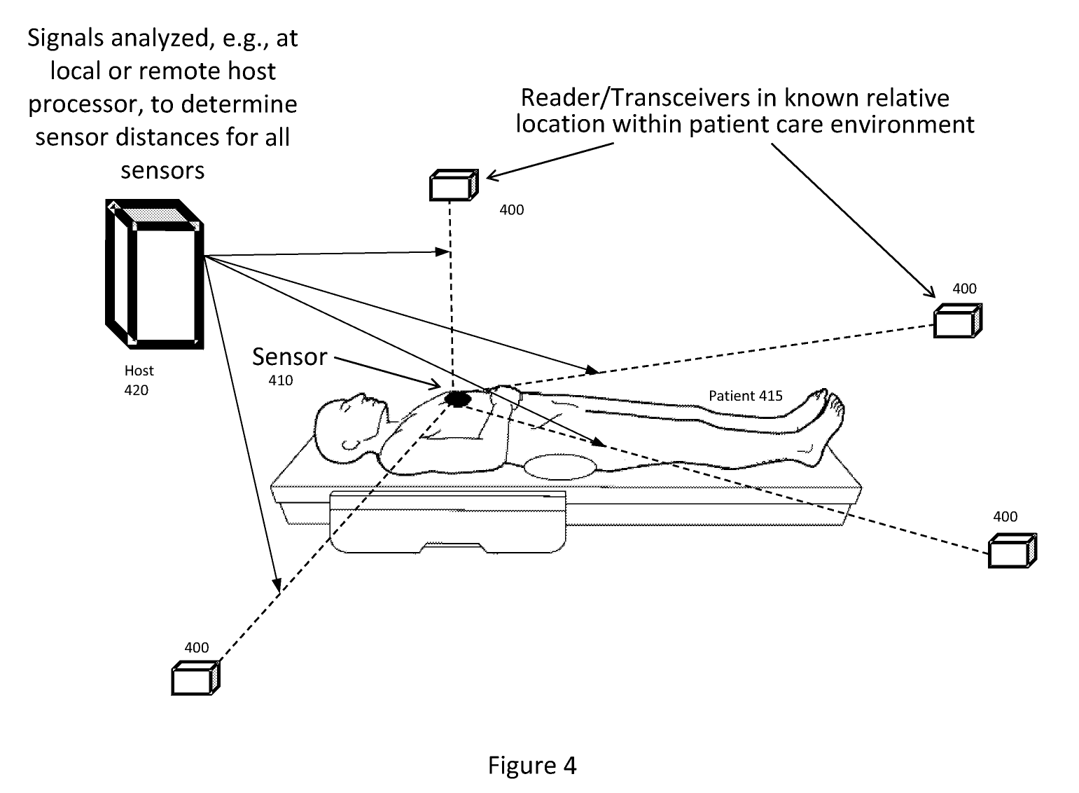

FIGS. 2A-2C illustrate some possible arrangements where three sensors 200 (FIG. 2A) and two sensors 205 (FIGS. 2B and 2C) are placed on the user. FIG. 4 illustrates the detection of each sensor's location within the monitored environment; for clarity, only a single sensor is show in FIG. 4. In the case of three sensors, if the location of each of the 3 sensors is known, the rotation of the user about the cephalo-caudal axis, as well as the tilt of the user, can be determined. The sensors on either side of the user's hip can be used to determine the rotation about the cephalo-caudal axis. For example, if the left sensor is located above the right sensor, the user is rotated onto the right side of their body. Alternatively, if the right sensor is located above the left sensor, the user is rotated onto the left side of their body. If the sensor that is closer to the head is located above a sensor(s) that is closer to the hips or feet, then the user is tilted head up, or in a reverse Trendelenberg position. If a sensor that is closer to the head is located below a sensor(s) that is closer to the hips or feet, then the user is tilted head down, or in a Trendelenberg position. Although three sensors 200 are shown in FIG. 2A, more than three sensors can be used in a similar fashion, where at least one sensor is preferably located more to one side of a patient's body than another sensor and at least one sensor is located more toward the head of a patient's body than another sensor.

If only side-to-side rotation information is desired, then only two or more sensors can be used, with at least one sensor being more associated with a particular side of the body (i.e. left/right or cephalo/caudal) than another sensor. This arrangement is illustrated in FIGS. 2B and 2C. If the left sensor 210 is above the right sensor 215, the user is rotated to the right side. If the right sensor is above the left sensor, then the user is rotated to the left side. Similarly, if only head-to-foot tilt information is desired, then two sensors can be used as shown in FIG. 2C, with one sensor 220 located closer to the head than another sensor 225.

It is also possible to determine both the side-to-side rotation (rotation about the cephalo-caudal axis) and the head-to-foot tilt (rotation about the transverse axis) of the user using two or more sensors. If, as shown in FIG. 2C, the sensors are placed with one sensor closer to one side (along the transverse axis) than another and with one sensor closer to the head (along the cephalo-caudal axis) than another, then knowing the three dimensional location of each sensor relative to each other in isolation or combination with the three dimensional location of each sensor relative to bed will give both left and right rotation (about the cephalo-caudal axis) and tilt (about the transverse axis) information. If for instance, the user is rotated onto their right side, the sensor closer to the right side will be lower compared to when the patient was lying flat relative to the bed. Each sensor will also be closer along the right-to-left (transverse) axis of the bed to the other sensor, but it will not be closer along the head to toe (cephalo-caudal) axis of the bed to the other sensor. Similarly if a user is tilted with his/her head up, the sensor closer to the head will be move up along the gravitational axis more than the other sensor and each sensor will be closer along the head-to-toe (cephalo-caudal) axis of the bed to the other sensor, but it will not be closer along the right to left (transverse) axis of the bed to the other sensor. If both tilt and rotation are occurring, then one sensor will be higher and both the distance between the sensors along the bed's head-to-toe (cephalo-caudal) axis and along the bed's left-to-right (transverse) axis will change. This information can be used in part or in its entirety to determine one or more of the following: whether the user is tilted (rotation about the transverse axis) and rotated left or right (rotation about the cephalo-caudal axis), the direction of tilt and rotation, the degree of tilt and rotation, and the angular velocity. In the case of a pure tilt or a pure left or right rotation, the information about the height difference between the sensors may be sufficient to determine the degree of tilt or rotation. If there is a combination of both tilt and left or right rotation, both the height difference between sensors and the changes in distance, if any, along the right to left (transverse) axis and head-to-toe (cephalo-caudal) axis of the bed can be used alone or in combination to determine the degree of tilt and left or right rotation.

In multi-sensor embodiments, the sensors are preferably sufficiently separated from one another on the body that the system is able to identify that there is separation between or among the sensors. The minimum separation distance can vary with the embodiment, and in particular the type of sensor used. Essentially, the system needs enough spatial resolution to determine how the sensors are located relative to at least one of each other or the environment. If the location sensing system is able to resolve that at least two sensors are in separate locations, then the sensors are generally spaced sufficiently apart.

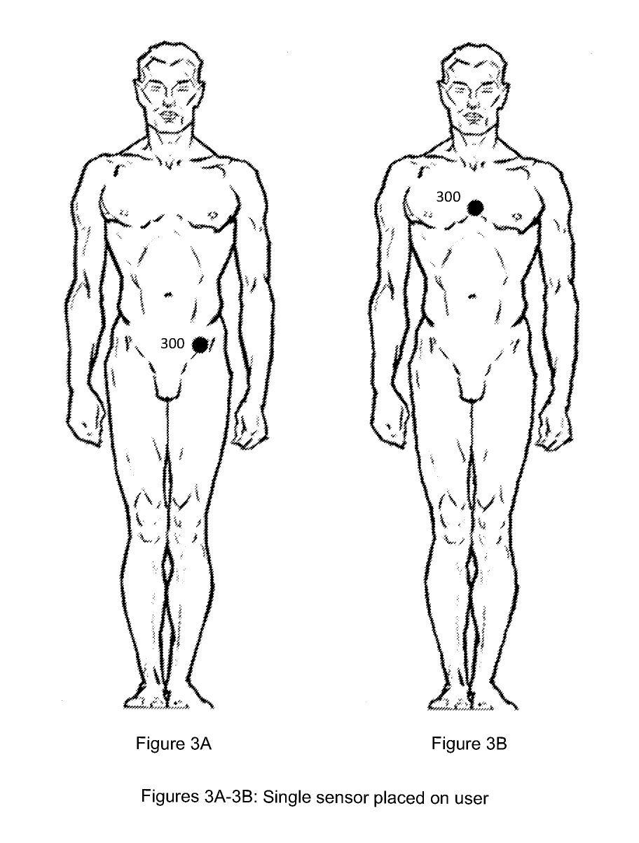

FIGS. 3A-3B show embodiments of the system where only a single sensor unit 300 is placed on the user. FIG. 4 again shows the detection of sensor location within the monitored environment, where each of the transceivers 400 senses the location of the sensor 410 on a patient 415 and communicates with a host 420. The communication can be either wired or wireless, and the host can be either remote or local. The transceivers 400 can, but need not each operate on the same technique. While four transceivers are illustrated in FIG. 4, the number of transceivers required depends upon the information to be gathered and the type of sensing device being used. In general at least 3 transceivers are used. In an embodiment, the host analyzes the received signal strength of the various transceiver signals, although numerous other techniques are also acceptable, as discussed above. The transceiver units themselves can also analyze the signals and otherwise take on many of the tasks of a host and processing unit. The transceivers in some embodiments will be in a fixed location. In other embodiments, the transceivers can be moveable, as long as their location within the monitored environment, or as long as their location relative to a given point of reference, such as a hospital bed, is known.

The individual sensor unit can contain one or more sensors. Multiple sensors can be placed within a single housing structure, as long as there is sufficient separation of with respect to the system's special resolution the sensors within the housing structure. FIG. 3B shows a single sensor on the sternum of a user. Here the sensor unit can contain multiple sensors that are sufficiently placed apart, such that the system can resolve that there are multiple sensors present and sense the relative distances between them. The spatial relationship between the individual sensors within the sensor unit is typically known. The same methods as described above for determining user orientation and tilt using multiple sensors can then be used.

FIG. 3A image shows a single sensor unit on one side of the user. Here the sensor unit is located on the pelvis as an exemplary placement, but essentially any placement on the torso can be adequate for many embodiments, while placement on the limbs is appropriate for other embodiments seeking to develop other information.

In some embodiments, the sensor unit can contain only a single sensor and still be able to determine left-right rotation. When placed sufficiently to one side of the user, the sensor location along the vertical axis (up-down axis or parallel to the direction of gravitational acceleration) changes depending on the rotation of the user. In FIG. 3A, the sensor is placed on the left side of the user's pelvis. If the user is flat, the sensor is at a given height. If the user is rotated with the left side up, the sensor is at a higher height than when the user is flat. If the user is rotated with the left side down, the sensor is at a lower height than when the user is flat.

Although FIGS. 3A-3C show the sensors placed on the sternum and the pelvis, and FIGS. 2A-2C show similar placements, the system of the present invention does not require that the sensors be placed in these locations. In determining tilt and rotation, separation along the left-right axis of the user and the cephalo-caudal axis of the user respectively are useful, but the exact location is not critical. The sternal location of a sensor may be useful for easy placement of the sensor in a location on the body that may be easy to access, does not contain a lot of tissue, and is not excessively moist, and is not exposed to a lot of friction. The sensor in the sternal region may also be useful in determining the heart rate and breathing rate, among other things. If the location of the sensor can be detected with sufficient spatial resolution, small movements of the sensor caused by cardiac or respiratory activity can be detected. Alternatively, other sensors such as accelerometers, electrodes, gyroscopes, magnetometers, impedance sensors can be used additionally, and can even be placed in the same sensor unit, to help further resolve the position and orientation of a patient and also determine such measures as heart rate and breathing rate. Sensors placed on the pelvis are located such that they provide information closely related to the rotation of the pelvis, which is useful in pressure ulcer management, where pelvic pressure ulcers are common.

Similarly sensors may be placed elsewhere on the body giving different information. For example, sensors can be placed on the limbs and head to determine their location, movement, and orientation. Sensors may also be placed in the environment to determine the location, orientation, and movements of users relative to objects in the environment. The support surface or specific areas of the support surface can have sensors to determine the location of users relative to a support surface. Sensors may be placed on any asset where it may be desirable to understand the location, position, orientation and other characteristics of such asset relative to the environment. In addition to determining the relative location and/or orientation of patients and objects, sensors may also be placed on floors, walls, elevators, etc. to define the physical architecture of the patient care environment. In such a manner, the location of patient's relative to the floor and other structures can be determined. Alternatively, the physical architecture of the patient care environment can be pre-programmed into the system.

Though the term sensor is used in this document, the objects placed on or near the user in known locations relative to areas of the user's body can be sensors or markers, and the term sensor is intended to encompass both as appropriate. For instance, the objects can be emitters of signals taken from a list comprising light, acoustic, mechanical, and electromagnetic. The objects can also be reflectors or visual markers. The markers can be placed on or near the user or they can already exist on or near the user. For example, machine vision or other computational techniques can be used to track elements on or near the user. The main point is that the location and/or orientation of these objects within the monitored environment can be determined using the apparatus and methods described herein.

It should be further pointed out that the sensors described herein developed for position, orientation, and location sensing can also be incorporated into other devices. For example, standard EKG electrodes can be designed to accommodate the sensing devices and methods described in the present invention. EKG electrodes are necessarily positioned on the patient's body in known locations relative to each other and the user. With EKG electrodes that have been outfitted with the location sensing apparatus of the present invention, the relative location of the sensing electrodes will be known. In such a manner, the EKG electrodes will provide data regarding both electrocardiac activity as well as the location, position, and orientation data of the patient.

Location-Based Bed Exit Sensing

Detecting patient bed exits is important for patient care facilities as well as home care. Movement of the user and relocation beyond a certain predefined threshold amount can be interpreted to be a bed exit. Thresholds in movement can be used to determine limits to define a bed exit. Similarly, the bed or support surface location can be known by the system: it can be programmed in, sensed with sensors, or determined from normal user movement/relocations. The location of the user relative to the support surface can be used to signal a bed exit. Different thresholds of movement, or degree of relocation relative to a support surface, can be set. For example, if the user has lines, catheters, or drains in place, such that they are physically tethered to a specific location, a lower threshold may be set to signal that a line, catheter or drain may be at risk of being dislodged. Alarms or other information can be given to the user or patient care workers. The information can also be stored. Movement information can be analyzed to promote or warn against movement.

Location-Based Fall Sensing

A fall is another important event for patient care facilities and home care. Several characteristics of location can be used to detect a fall. The location areas of the body that do not normally come close to the ground (such as the torso, head, shoulders, etc.) can be sensed and if these areas are determined by the sensing system to be close to the ground, the likelihood of a fall is greater. The speed of movement of sensors associated with a user can also be analyzed and interpreted over time to determine the speed that the user, or areas of the user's body, are falling. Though not all falls are fast, fast downward motion of certain areas of the body may indicate an increased likelihood of a fall. Similarly, characteristic falling movements or positions of body parts during or after a fall may be sensed by one or more sensors that are associated with specific areas of the user's body. For example, a sensor on an arm and torso may allow for sensing of an outstretched arm during a fall. Similarly, determining that the user is lying in a location that the user would not normally lie down, such as away from the support surface, bathtub, chair, bed, or other location associated with normally lying flat, may also indicate an increased likelihood of a fall. One or more sensors can be used to determine if the user is lying down. The sensing apparatus of the present invention can also be designed to incorporate an accelerometer, magnetometer, gyroscope, barometer, and other sensors to help refine the analysis of positions, orientations, and movements that are characteristic of a fall.

One or more of these techniques can be used to determine the likelihood of a fall, and combining them can increase the sensitivity and/or specificity of the detection. The location sensing system can also be combined with sensing of other physiologic characteristics from a group comprising orientation, heart rate, respiratory rate, temperature, blood pressure, pulse oximetry, capnography, and blood glucose levels.

The user can also be monitored over time to determine common, typical or normal movements and therefore any abnormal movements can be flagged, inspected more closely, logged differently, or can trigger alerts or information to be sent to relevant care providers. A sensed fall, or the determinants or likelihoods of impending falls, can be transmitted to care providers.

Live Tracking of Patient Position During Procedures and Diagnostics

Monitoring the orientation and movements of a patient can be important during diagnostic and therapeutic interventions. The system described herein can be used to indicate when a patient movement has compromised a diagnostic or therapeutic intervention. Alternatively, the system can also be used to help guide diagnostic and therapeutic interventions based on the perceived or detected location, position, orientation, and movements of a patient. Data regarding the patient's overall orientation, or specific regions of a patient's body can be provided to guide diagnostic and therapeutic interventions. In one example of use, the system of the present invention can be used to monitor a patient's respiratory cycle, and diagnostic or therapeutic interventions can be timed according to the patient's respiratory cycle.

Incorporating Other Sensors

The sensing system of the present invention can be designed to incorporate information from accelerometers, gyroscopes, magnetometers, barometers, and other sensors. The information provided from these additional sensors can be used to increase the sensitivity and specificity of detecting patient location, orientation, and movements.

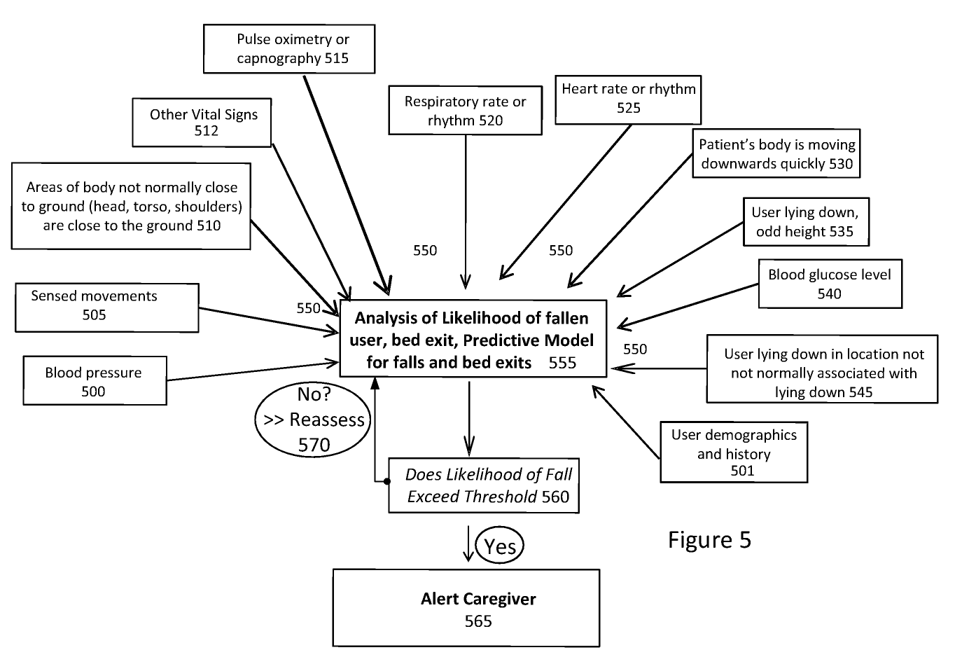

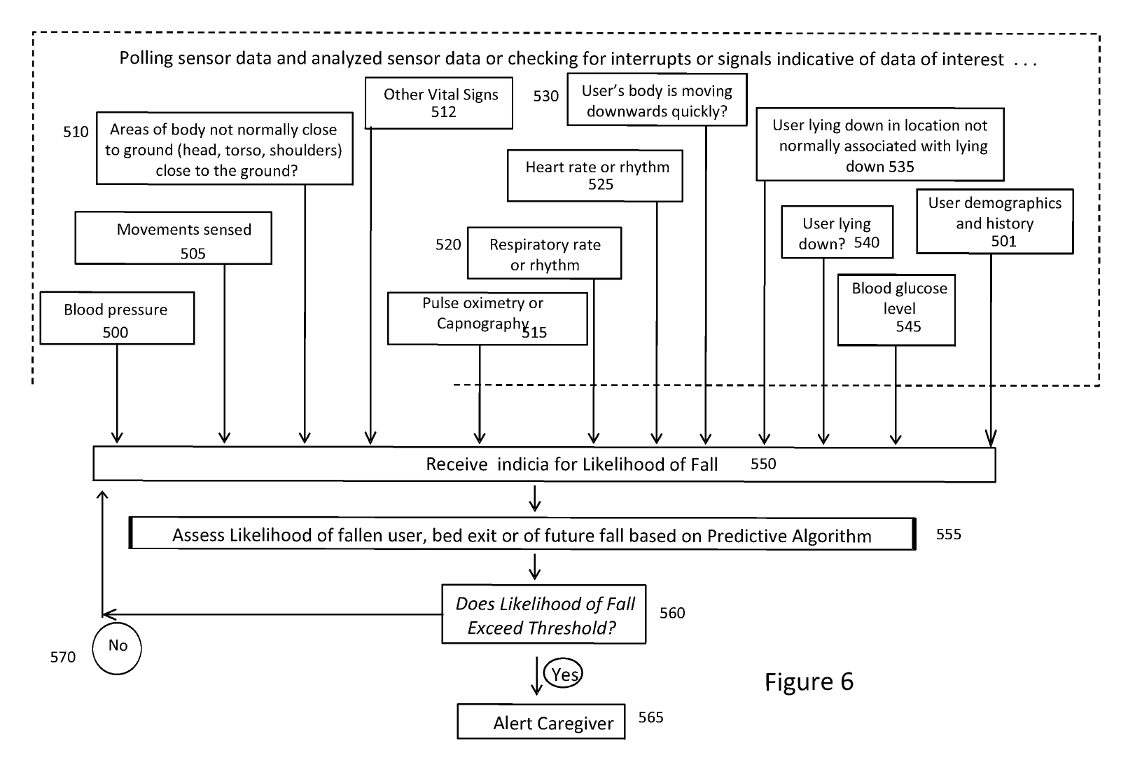

Referring next to FIGS. 5 and 6, an example of the signal processing of a system for detecting patient position, location and movement, such as orientation, falls, and bed exits, and live tracking, is shown. In particular, a variety of patient characteristics are monitored by the system as described in connection with FIG. 4. Those characteristics are compared to predetermined “normal” values, which can be patient specific or generalized to a larger sample. If the monitored characteristics fall outside the expected range, an interrupt or flag is generated, which then increments an indicia that is evaluated by the program running in the host processor to determine whether that characteristic, either alone or taken in combination with other data from the monitored characteristics, exceeds an overall threshold. The overall threshold, like the thresholds for the various characteristics, can be patient specific or more general across a predetermined population. If the overall threshold is exceeded, a caregiver is alerted.

Thus, with specific reference to FIG. 5, representative sensed patient characteristics can comprise blood pressure as indicated at 500; sensed abnormal movements indicated at 505; areas of the body proximate to the ground (such as head, torso, shoulders) that normally are not close to the ground as indicated at 510; various other vital signs as indicated at 512, and 501 user demographics and history, including patient acuity, diagnoses, medical history, history of falls or pressure ulcers, Braden score, medications, and age, weight, gender, other patient specific variables; pulse oximetry or capnography levels as indicated at 515; respiratory rate or rhythm as indicated at 520; heart rate or rhythm as indicated at 525; patient's body detected as moving downward quickly as indicated at 530; patient lying down at abnormal height as indicated at 535; blood glucose level as indicated at 540; and user lying down in abnormal location as indicated at 545. A predictive algorithm can assign different weighting to the various characteristics when being evaluated by the host program for determining whether the overall threshold is exceeded. For example, if areas of the body are abnormally close to the ground (510), that characteristic can be assigned a weight greater than an abnormal blood glucose level (540).

Once the various patient characteristics are detected and received as indicated at 550, FIG. 5, the totality of the detected variables is factored and analyzed to determine the likelihood of a fall, or a bed exit, or a movement, as indicated at 555. The result is compared to a predetermined threshold as discussed above, as shown at 560. If the result exceeds that threshold, a caregiver is notified as shown at 565. If not, the process loops at 570 to reassess any detected conditions, essentially providing continuous monitoring. In an alternative embodiment, any single variable (or combination of specific variables) which exist in patterns associated with increased likelihood of falls or bed exits or movements can set a flag which causes the overall threshold to be exceeded, in which case a caregiver is notified.

Referring next to FIG. 6, the process of FIG. 5 is illustrated in a somewhat different form, with like reference numerals since the underlying structure of the control program and host is the same. Each of the monitored characteristics is monitored by the host running the control program, and the sensed characteristics are accumulated and assessed by a predictive algorithm to assess the likelihood of a fall or bed exit. Also, as discussed above, in an alternative embodiment, any characteristic that is detected to be outside of acceptable limits can increment or otherwise factor into a measure or risk, or generate an interrupt, or flag. The messages, interrupts or flags each increment or otherwise are combined to yield an indicia used to assess the likelihood that the monitored patient has either fallen, or moved in a way that generates concern, or made a bed exit. The remainder of FIG. 6 operates as discussed above in connection with FIG. 5.

Alternatively, the values independently, or in combination, may suggest an abnormal state or a state worthy of alarm even if they are within normal limits. For example, a blood pressure that is considered normal in the supine position may be considered abnormal when the user is standing up. The detection of such patterns of combination of values may also be used to increase the level of alarm or alert a caregiver.

The Predictive Algorithm illustrated in FIGS. 5 and 6 takes in information from the different sensors and data about patients to assess the likelihood of a potential fall or bed exit. Depending upon the desired implementation, budding and iterating the algorithm can make use of elements from Machine learning techniques, Regression Models, and other techniques known in the art. Patterns in the data that are associated with a fall, bed exit, or future fall can be used. Data can be entered of the occurrence of falls or bed exits along with the input data to help build the algorithm. Similarly, certain inputs, such as location data, acceleration data, or other inputs or combinations thereof that are highly associated with falls or bed exists can also be used to help build the algorithm. Such data, as discussed earlier, may include the location of the patient and parts of the patient close to the ground, etc. An alternative that can be used in addition to or independently from an algorithm based on statistical techniques is one that can be expertly generated. Such an algorithm may include detecting unsteady movement patterns, low blood glucose, low blood pressure, and other data associated with unsafe ambulation.

Referring next to FIGS. 7 and 8, an embodiment of a sensor suitable for use with the present invention is illustrated. As shown in FIG. 7, one presently preferred form of a sensor 700 comprises a multi-axial accelerometer 705 with associated processor 710 and related electronics. One acceptable accelerometer is the type LIS344ALH three axis accelerometer available from ST Microelectronics, although sensing on three axes is not required in all embodiments. Many other acceleration sensors would suffice in this role, and would be known to those knowledgeable in the art. In addition to the accelerometer, the sensor 700 can, depending upon the implementation, also comprise a capacitive sensor 315, a blood pressure sensor 720, a sensor for other vital signs 712 and a sensor or means for input of associated patient characteristics, including patient acuity, medications, Braden score, etc., a blood glucose sensor 715, and a pulse oximeter sensor 730. The microprocessor 710 can comprise a built-in ND converter and stored sensor identifier, and communicates with a base station/host 735 which can include a transceiver for wireless communications, located near enough to reliably receive wired or wireless signals, through an RF transceiver 740 and antenna 745. Alternatively, the transceiver/base station 735 communicates with a remote host. In either case, the host can ultimately link to terminals (not shown) or other means for communicating alerts to a caregiver. The terminals or other alert mechanisms can be, for example, integrated into the patient sensor or support system, in the patient room, at the nursing station, or at other locations. It will be appreciated that, while not shown, a battery or other power source is provided in the sensor 700. It will be appreciated by those skilled in the art that the functions of the host can reside in several different locations in a system in accordance with the present invention. For example, the host functionality can largely reside in the sensor itself, or that functionality can coexist within the base station, or it can be external to both, or the functions can be split across multiple devices. Likewise, the functions of the sensor of FIG. 7 can be split across a plurality of devices. FIG. 8 illustrates an embodiment of a sensor 800 having indicia 805 for assisting in placement of the device on the patient in a predetermined orientation.

Having fully described a preferred embodiment of the invention, and numerous aspects thereof, as well as various alternatives, those skilled in the art will recognize, given the teachings herein, that numerous alternatives and equivalents exist which do not depart from the invention. It is therefore intended that the invention not be limited by the foregoing description, but only by the appended claims.

We claim:

1. A system for analyzing the movement, orientation, and location of a user within a known environment comprising one or more sensors affixed to a user, a plurality of remote devices positioned in a known relative location within the known environment and in communication with the one or more sensors, and a processor for analyzing data received from the one or more sensors representative of patient characteristics comprising at least one of user position relative to the cephalo-caudal, transverse and anterior-posterior axes of the user and user position within the known environment.

2. A method for analyzing the movement, orientation, and location of a user within a known environment comprising positioning one or more sensors in approximate predetermined locations on the body in an approximately known orientation, positioning a plurality of remote devices in known relative locations within the known environment, and analyzing data received by the remote devices from the one or more sensors in accordance with a predictive algorithm to identify user actions or characteristics that exceed a predetermined threshold indicating likelihood of at least one of a fall, bed exit, or other movement of concern.
